(19) United States
(12) Patent Application Publication
(10) Pub. No.: US 2004/0162292 A1
(43) Pub. Date: Aug. 19, 2004

(54) Multivitamin formulations for promoting healthy collagen, and methods of their use

(76) Inventors: Kenneth L. Evenstad, Naples, FL (US); Lori M. Freese, Monticello, MN (US); Harish K. Pimplaskar, Durham, NC (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 10/725,692

(22) Filed: Dec. 2, 2003

Related U.S. Application Data
(60) Provisional application No. 60/430,531, filed on Dec. 3, 2002.

(51) Int. Cl. A61K 31/525

(52) U.S. Cl. 514/251

(57) ABSTRACT

Described are vitamin and mineral formulations comprising folic acid compound in amounts to promote healthy collagen, and optionally other vitamins and minerals such as vitamin B₆, vitamin B₁₂, calcium, magnesium, boron, as well as methods of administering such formulations to promote healthy collagen, to treat or prevent collagen-related conditions, and to facilitate tissue healing.

This non-provisional application claims priority to U.S. provisional application serial No. 60/430,531, filed Dec. 3, 2002, entitled MULTIVITAMIN FORMULATIONS FOR PROMOTING HEALTHY COLLAGEN, AND METHODS OF THEIR USE, which Application is incorporated herein by reference in its entirety

### FIELD OF THE INVENTION

The invention relates to administering folic acid compound-containing formulations to individuals to facilitate healthy collagen production, to thereby treat or prevent conditions related to inadequate or unhealthy collagen production or to facilitate healing or production of healthy collagen-containing tissue. In exemplary embodiments the invention relates to periodically administering multivitamin formulations that contain amounts of folic acid to facilitate healthy collagen production, optionally in combination with vitamin B₁₂ or vitamin B₆, and optionally in combination with other vitamins or minerals.

### BACKGROUND

Multivitamin formulations are popular for supplementing dietary vitamins and minerals. Many varieties of multivitamin formulations are available, and some contain different combinations and amounts of two or more of folic acid, vitamin B₁₂, vitamin B₆, vitamins A, C, D, E, K, and minerals such as boron, calcium, magnesium, iron, etc.

Certain multivitamin formulations are described as useful for administering to individuals to treat or prevent various specific medical conditions. Examples of these conditions include osteoporosis, skin conditions, stress, cardiac health, promoting general health and dietary supplementation to pregnant women, to name only a few. Multivitamin formulations designed for such specific purposes typically include selected amounts of specific vitamins and minerals believed to be of particular value to individuals having the stated conditions. As a single example, multivitamin tablets containing folic acid, vitamin B₁₂, and vitamin B₆ are sold to be taken daily to reduce serum homocysteine and promote cardiovascular health. Other examples of popular multivitamin formulations sometimes contain vitamins and minerals including calcium or magnesium, and may be taken for treating or preventing osteoporosis.

### SUMMARY

Recently, in the context of vascular disease, a relationship has been proposed relating the amino acid homocysteine to certain collagen (see, e.g., Homocysteine and Cardiovascular Health, Heart Health Infocenter, Holisticonline.com, http://holistic-online.com/Remedies/Heart homocysteine.htm, “Homocysteine is thought to promote atherosclerosis by directly damaging the artery and by reducing the integrity of the vessel wall, . . . as well as by interfering with the formation of collagen (the main protein in bone)). According to the invention, it is thought that the presence of excessive serum homocysteine could hinder or prevent healthy collagen production or the maintenance of healthy levels of biological collagen.

Separately, metabolization of homocysteine is thought to be important for production, formation, and maintenance of collagen in the body because sulfur from a metabolized homocysteine molecule is used in building collagen.

A variety of, i.e., at least 19, separately identifiable forms of collagen are recognized and known to be distributed in tissues including bone, tendon, skin, smooth muscle, arteries, vascular tissue, teeth, ligaments, and fetal and adolescent skeleton. (See Liu et al., “Collagen in Tendon, Ligament, and Bone Healing,” Clinical Orthopaedics and Related Research, 318 265-278 (1995)). Collagen is also believed to have a role in healing surgical wounds. (See Harrison's Principles of Internal Medicine, 13^{th} ed., p. 445).

According to the present invention, a variety of medical conditions referred to herein as “collagen-related conditions,” are believed to relate to reduced, inadequate, or unhealthy biological amounts of collagen. These conditions may be caused by reduced amounts of healthy collagen in the body due to one or more biochemical conditions or genetic abnormalities in an individual that prevent or hinder the body's ability to produce or maintain collagen e.g., caused by an unhealthy or inadequate diet. According to the invention, such conditions may be treated or prevented by administering multivitamin formulations containing folic acid and other optional vitamins and minerals, in amounts effective to promote or enhance collagen production, promote the proper formation of collagen, or maintain healthy levels of collagen in the body. According to the invention, the formulations containing folic acid can be administered not only to promote collagen production in individuals who have inadequate collagen levels, but also to augment normal collagen production and to maintain the health of bodily tissues having collagen, such as skin, muscles, bones, tendons, ligaments, cartilage, and other types of tissues that contain collagen. The invention therefore relates to methods of periodically orally administering folic acid and other optional vitamins and minerals in amounts effective to promote healthy collagen production and maintain healthy levels of biological collagen, thereby preventing or treating collagen-related conditions or otherwise promoting or sustaining the health of collagen-containing tissues.

In specific embodiments, the invention provides folic acid-containing formulations and methods of administering the formulations for the treatment or prevention of conditions that are or can be caused by collagen deficiencies or unhealthy or defective collagen production. The multivitamin formulations can also be used to generally promote healthy collagen production and to generally promote production of healthy tissue or maintain healthy tissue having collagen. Individuals having conditions that are either caused by a deficiency in collagen or conditions related to unhealthy or defective collagen production, or that do not necessarily have a collagen deficiency but who have a condition that can benefit from improved collagen availability (e.g., individuals who are or may be pregnant, who have a skin condition, who have a degenerative bone or skin condition, or who suffer from a tissue wound or tissue condition) are sometimes referred to herein as having a “collagen-related condition.” Thus, the inventive methods do not require that a collagen deficiency or acute medical condition be present for administration of the folic acid-containing formulations described herein. The inventive methods contemplate prophylactic or preventative treatment of any collagen-related condition by administering the described formulations. The inventive methods also contemplate administering the described formulations to augment normal collagen production for the purpose of further promoting or maintaining healthy bodily tissue such as healthy skin, bone, muscles, tendons, cartilage, ligaments, and for promoting healthy fetal bone, skin, or other developing tissue having collagen or wherein the development is affected by the presence of collagen.

Exemplary collagen-related conditions are believed to include: general collagen deficiency or inhibited, abnormal, or otherwise defective or inadequate production of collagen (e.g., caused or exaggerated by homocysteine or a body's inability to metabolize homocysteine); general and specific degenerative conditions such as degenerative bone disease, osteoporosis, Alzheimer's disease, and arthritis; tissue wounds such as muscle, skin, bone, or ligament wounds; pregnancy and conditions related to pregnancy nutrition, including general nutritional and dietary needs for proper fetal development; and skin conditions, e.g., acne, psoriasis, rash, etc.

Embodiments of the inventive methods include administering folic acid-containing formulations for: treatment or prevention of general collagen deficiency or conditions where collagen production is inhibited; treatment or prevention of degenerative bone disease such as osteoporosis; treatment or prevention of other degenerative disease such as Alzheimer's disease and arthritis; treatment of wounds or promotion of wound healing including skin wounds, muscle wounds, bone wounds, cartilage or ligament or other tissue wounds, including healing such wounds generally or for administering the formulations prior to or after surgery; promoting prenatal health and nutrition (and fetal development); and treating or preventing skin conditions and promoting healthy skin. Preferred inventive methods include the oral administration of folic acid-containing formulations.

The inventive formulations and methods may further relate to administering amounts of folic acid compound that exceed Dietary Reference Intakes (“DRIs”) conventional for folic acid compounds. While folic acid compounds are known and administered for various conditions, many commercially available multivitamin formulations continue to be taken to provide daily dosages of folic acid compounds that may be below amounts required for the treatment of collagen-related conditions or for maintaining healthy collagen levels in the body. Therefore, conventional vitamin formulations may fail to provide adequate or optimal supplementation of dietary folic acid, and consequently may not sufficiently promote or facilitate healthy collagen production or otherwise be in a quantity sufficient to treat or prevent a collagen-related condition.

The inventive formulations and methods provide relatively high daily dosages of folic acid when compared to certain commercially available multivitamin formulations of similar compositions and uses, which can be administered to individuals having collagen-related conditions or to facilitate healing or production of collagen-containing tissue. The inventive formulations preferably include at least or greater than 400 micrograms (mcg), and more preferably at least 500 mcg, 1000 mcg, 1500 mcg, or 2000 mcg of a folic acid compound per dose. Such formulations having these quantities of folic acid compound can be referred to herein as “dose formulations” or “unit dose formulations” intended to be administered or taken as a single dose. In some formulations, depending on the collagen-related condition being treated, the folic acid compound can be present up to and including about 10 milligrams. The inventive methods provide for the administration of dosages greater than 400 mcg of folic acid compound per day, and in preferred embodiments, greater than 500 mcg, greater than 1 mg, 1.5 mg, and greater than 2 mg folic acid compound per day.

In some embodiments, the inventive formulations can include vitamin B₆ in a useful amount, e.g., effective for the for the treatment of a collagen-related condition or for maintaining healthy collagen levels in the body, for example from about 5 or 10 to about 50 milligrams. Vitamin B₆ is a cofactor in metabolizing homocysteine, and therefore can reduce serum homocysteine and further promote the production and presence of healthy collagen.

In some embodiments, the inventive formulations can include vitamin B₁₂ in a useful amount, e.g., effective for the for the treatment of a collagen-related condition or for maintaining healthy collagen levels in the body, for example from about 15, 20, 50, 100, or 150 micrograms vitamin B12 up to about 1000 micrograms of vitamin B₁₂. Vitamin B₁₂ is also a cofactor in metabolizing homocysteine, and therefore can also reduce serum homocysteine and further promote the production and presence of healthy collagen.

In one embodiment the invention provides formulations that specifically contain or consist of compounds that are effective for the treatment of collagen-related conditions or for maintaining healthy collagen levels in the body, optionally with other inert ingredients, for example, surfactants, cosolvents, and excipients. These may include formulations consisting of a folic acid compound, a vitamin B₆ compound, and a vitamin B₁₂ compound in amounts sufficient for the treatment of collagen-related conditions or for maintaining healthy collagen levels, and optional inert ingredients. In these formulations, the amount of the folic acid compound can be at least 400 mcg, the amount of the vitamin B₆ compound can be in the range of about 5 or 10 to about 50 milligrams, and the amount of vitamin B₁₂ compound can be in the range of about 15, 20, 50, 100, or 150 micrograms vitamin B₁₂ up to about 1000 micrograms. These formulations can be particularly useful, for example, when the administration of vitamins or minerals other than folic acid, vitamin B₆, and vitamin B₁₂ is not necessary or is not indicated. The invention also contemplates methods of administering these formulations for the treatment of collagen-related conditions or for maintaining healthy collagen levels.

In other embodiments, the inventive formulations can include amounts of other vitamins and minerals including, but not limited to, calcium, magnesium, thiamine, riboflavin, boron, iron, vitamin A, vitamin C, vitamin D, vitamin E, vitamin K, other useful or similar substances, and combinations thereof. Multivitamin formulations as described herein do not include aspirin, and preferably do not include any other substances that would require regulatory approval of the sale or use of the folic acid compound-containing formulation.

Vitamins and minerals included in the folic acid compound-containing formulations can be selected for administering to treat or prevent specific conditions. For example, multivitamin formulations of the invention formulated and administered to treat or prevent degenerative bone disease such as osteoporosis, other degenerative tissue disease such as arthritis, Alzheimer's disease, or other degenerative conditions, in addition to containing folic acid compound, may include vitamin B₆, vitamin B₁₂, and other vitamins and minerals useful to treat or prevent such conditions, including one or more of calcium, magnesium, vitamin D, boron, or others. When compared to commercial multivitamins formulated for treatment or prevention of these or similar conditions, the inventive formulations can be formulated to provide higher dosages of one or more of these vitamins or minerals, for example a dosage of folic acid compound greater than 1 mg or 2 mg per multivitamin dosage form or per day; a dosage of boron greater than 150 or 200 mcg, e.g., greater than 500 mcg, or in the range from 1 mg to 5 mg per day, or any amount up to 20 mg boron per day; a dosage of vitamin B₆ greater than 1.7 mg per dosage form or per day, preferably greater than 3 mg, vitamin B₆ especially from about 5 or 10 mg vitamin B₆ to about 50 or 100 mg vitamin B₆ per dosage form or per day; a dosage of vitamin B₁₂ greater than 25 mcg per dosage form or per day, e.g., from about 50 mcg, 100 mcg, or 150 mcg up to about 1000 mcg vitamin B₁₂ per dosage form or per day; greater than 250 mg and up to about 1000 mg calcium per dosage form or per day; and greater than 125 mg and up to about 250 mg magnesium per dosage form or per day. Therefore, in some embodiments, active ingredients of the formulation consist of a folic acid compound, a vitamin B₆ compound, a vitamin B₁₂ compound, calcium, magnesium, vitamin D, and boron, in amounts described herein.

In another embodiment, formulations can be prepared to improve pre-natal health, promote fetal development, or to supplement a pre-natal diet. These formulations include folic acid, and can also include vitamin B₆, vitamin B₁₂, and other vitamins and minerals useful to improve pre-natal health or to supplement a pre-natal diet, including one or more of thiamine, riboflavin, calcium, iron, magnesium, or others. When compared to DRI values for pregnancy or commercial multivitamins formulated for pre-natal health and diet supplementation, the inventive formulations can be formulated to provide higher dosages of one or more of these vitamins or minerals. For example, the inventive methods and formulations can provide a dosage form or a daily dosage of a folic acid compound in an amount greater than 400 mcg, e.g., equal to or greater than 1 mg or 2 mg up to about 5 or 10 mg folic acid compound per day; a dosage of vitamin C in an amount greater than 90 or 120 mg, e.g., 130 or 150 mg or greater; a dosage of thiamine in an amount greater than 3 mg, e.g., 3.5 or 4 mg; a dosage of riboflavin in an amount greater than 3.4 mg, e.g., 3.5 or 4 mg or greater; a dosage of vitamin B₆ in an amount that is greater than 1.7 mg, e.g., greater than 3 mg, preferably greater than 5 mg or 10 mg, and up to about 50 mg; calcium in an amount in the range from about 200 to 1000 mg; iron in an amount greater than 18 mg, such as from 20 to 100 mg, e.g., from 30 to 60 mg.

In another embodiment, formulations for the treatment or prevention of skin conditions or to promote healthy skin, in addition to a folic acid compound, can include vitamin B₆, vitamin B₁₂, and other vitamins and minerals useful treat or prevent skin conditions or to promote healthy skin, including one or more of vitamin C, vitamin A, and vitamin E, or other vitamins or minerals. When compared to the DRI or commercial multivitamins formulated for pre-natal health and diet supplementation, the inventive formulations can be formulated to provide higher dosages of one or more of these vitamins or minerals. For example the inventive formulations may contain a dosage of a folic acid compound in an amount greater than 1 mg; a dosage of vitamin B₁₂ in an amount greater than 2.4 mcg, e.g., 100 or 200 mcg vitamin B₁₂ up to about 1000 mcg vitamin B₁₂; a dosage of vitamin C in an amount greater than 90 or 120 mg, e.g., 130 or 150 mg or greater, e.g., up to 300 or 500 mg vitamin C; a dosage of vitamin E in an amount greater than 33 IU or 60 IU, e.g., 100 or 200 IU up to about 300 or 500 IU; and a dosage of niacin in an amount greater than about 16 mg, e.g., from about 18 to 30 mg niacin.

In another embodiment, formulations that promote wound healing, e.g., skin, muscle, tendon, ligament, or bone wounds, including or other than wounds cause by a surgical procedure, include folic acid compound and can also include vitamin B₆, vitamin B₁₂, and other vitamins and minerals useful for promoting wound healing, including one or more of vitamin C, vitamin E, zinc, and other vitamins or minerals. When compared to the DRI or commercial multivitamins, the inventive formulations can be formulated to provide higher dosages of one or more of these vitamins or minerals. For example the inventive formulations may contain a dosage'of a folic acid compound in an amount in the range greater than 400 mcg, e.g., 1 mg or greater folic acid compound; a dosage of vitamin B₁₂ in an amount greater than 2.4 mcg, e.g., 50 mcg, 100 mcg, or 200 mcg vitamin B₁₂ up to about 1000 mcg vitamin B₁₂.

An aspect of the invention relates to a method of facilitating or maintaining collagen formation to treat or prevent a collagen-related condition. The method includes orally administering a formulation comprising greater than 400 micrograms of folic acid compound, preferably greater than 500 micrograms of folic acid compound, and more preferably greater than 1000 micrograms of folic acid compound.

In another aspect, the invention relates to a method of facilitating or maintaining collagen formation, the method including orally administering one or more formulation comprising greater than 400 micrograms folic acid compound, to provide a daily dosage of greater than 800 micrograms folic acid compound per day. The method can facilitate collagen formation to treat or prevent a condition such as a degenerative bone disease, osteoporosis, arthritis, or Alzheimer's disease; the method can facilitate collagen formation to treat or facilitate healing of a skin or muscle wound; the method can facilitate collagen formation to promote post-surgical skin or muscle tissue healing; and the method can facilitate collagen formation and promote pre-natal health or fetal development.

In yet another aspect, the invention relates to a method of treating or preventing degenerative bone disease, Alzheimer's disease, or arthritis, by orally administering a formulation comprising greater than 1 milligram of a folic acid compound.

In yet another aspect the invention relates to a method of improving pre-natal health or fetal development, by orally administering a formulation comprising from about 1.5 to 10 milligrams of folic acid compound.

Another aspect of the invention relates to a method of promoting healthy skin by administering a formulation comprising greater than 400 micrograms folic acid compound. The formulation can preferably contain, vitamin B₆, vitamin B₁₂, vitamin C, vitamin E, and boron.

Yet another aspect of the invention relates to a method of promoting post-surgical health or recovery by orally administering a dietary supplement formulation comprising from about 1 to about 10 milligrams folic acid compound and from about 20 to 1000 micrograms vitamin B₁₂.

Still another aspect of the invention relates to a method of promoting healing of a skin or muscle wound by orally administering a formulation comprising from about 1 to 10 milligrams folic acid compound and from about 20 to 1000 micrograms vitamin B₁₂.

### DETAILED DESCRIPTION

The invention relates to periodically administering a formulation that includes a folic acid compound to an individual in a daily dosage (e.g., daily forms or total daily amount) that promotes collagen production, promotes a healthy biological collagen level in mammals, especially humans, and promotes the formation and maintenance of healthy collagen-containing tissue in the body. In specific embodiments, the administration of folic acid-containing formulations can result in the prevention of collagen-associated disorders or can be used for the treatment of an acute collagen-related condition. Alternatively, the improved collagen production in the absence of a collage shortage or defective collagen production, or in the presence or absence of an acute medical condition, can still promote the healthy development or healing of tissue that relies on collagen, such as healthy skin, bones, muscles, ligaments, cartilage, or fetal tissue.

Collagen is the primary protein of connective tissue, which includes cartilage, bone, tendon, teeth, and skin. Collagen (in a pre-processed form called procollagen) is assembled in cells and consists of three polypeptides wound around each other in a triple helix form and are stabilized by intrachain disulfide bonds. After the helical molecule is assembled and modified in the cell it is secreted into the extracellular medium and further processed to a mature form (tropocollagen). Tropocollagen is a large molecule having a size of about 3,000 Å with a width of 14 Å. These matured collagen molecules assemble into fibrils in the extracellular space in a staggered, parallel, fashion wherein the molecules are stabilized in this fibril pattern by covalent cross-linking bonds between the N-terminus of one molecule and the C-terminus of another. The collagen fibrils further pack into hexagonal arrays to form fibril bundles. In tendons these collagen fibrils run parallel to the axis, in skin the fibrils are interlaced and branched.

Mammals have more than 30 genetically distinct collagen polypeptide chains, which make up the 19 different collagen variants that occur in different tissues. The most common are: Type I collagen, in skin, bone, tendon, blood vessels and cornea; Type II collagen, in cartilage, intervertebral disk; Type III in blood vessels, fetal skin. In one aspect of the invention the formulations described herein can facilitate or maintain collagen formation to treat or prevent a collagen-related condition. As used herein “collagen formation” is used in its broadest sense and refers to the production of collagen, its incorporation into collagen-containing tissue (including, e.g., the synthesis, processing, crosslinking, secretion, and assembly of collagen fibrils) and the presence of healthy collagen-containing tissue. “Facilitating collagen formation,” therefore, refers to the ability of a formulation described herein to positively affect the production of collagen, leading to the formation of healthy collagen-containing tissue in either physiologically normal (i.e. healthy) or disease states. Facilitating collagen formation may be brought about by the ability of the formulations described herein to promote steps, such as biochemical steps, leading to the formation of collagen fibrils. “Maintaining collagen formation,” refers to the ability of a formulation described herein to preserve existing healthy collagen-containing tissue, by for example, by preventing the degradation of collagen or by replenishing collagen to tissue wherein collagen is broken down by natural processes or disease-associated processes.

The term “folic acid compound” is used in the present description in a manner consistent with its understood meaning in the vitamin and medical arts. The term means compounds generally understood to provide vitamin (folic acid) efficacy in its administered form or as a derivative of its administered form. The term refers to compounds commonly referred to as folic acid, folinic acid, folacin, tetrahydrate folate, and pteroyl monoglutamic acid, and more generally includes all pteroglutamates having vitamin activity.

The daily oral dosage of folic acid compound administered according to the invention can preferably exceed daily recommended intakes (“DRIs”) that are conventional for folic acid compounds, for certain individuals and perhaps for certain individuals having specific conditions. The DRI for folic acid compound is 400 mcg, generally, and 600 mcg for pregnant women. Table 1 lists the DRI of folic acid compound as well as selected other vitamins and minerals. According to the inventive methods and formulations, daily oral dosages can exceed one or more of these DRIs. The inventive formulations can include, for example, folic acid compound in an amount greater than 400 micrograms (mcg), 500 mcg, 600 mcg, 1000 mcg, or 1500 mcg. The amount of folic acid compound administered daily or in a dosage form can sometimes depend on the collagen-related condition being treated, and can be in an amount in the range up to and including about 5 or 10 milligrams. Total daily dosage of folic acid compound, according to the invention, can be amounts greater than a DRI and as described herein, can be taken once in a single formulation dosage form or in two or more dosages at the same or different times throughout a single day.

TABLE 1
Recommended Intakes for Certain Vitamins and Minerals

| | | | | | UPPER LIMIT |
|---|---|---|---|---|---|
| NUTRIENT | CURRENT RDI* | | | NEW DRI** | (UL)*** |
| Calcium | 1,000 mg (adults); National Osteoporosis | | | 1,300 mg | 2,500 mg |
| | Foundation recommendation 1,200 mg daily. | | | | |
| Magnesium | NIH Recommends (Daily) | Men | Women | 420 mg | 350 mg (applies |
| | 18-30 years old | 400 mg | 310 mg | | only to intake from |
| | 30+ years old | 420 mg | 320 mg | | supplements) |
| Vitamin D | 400 IU (10 mcg) | | | 15 mcg (600 IU) | 50 mcg (2,000 IU) |
| | NIH Recommends (Daily) | Men | Women | | |
| | 19-50 years old | 5 mcg | 5 mcg | | |
| | 51-70 years old | 10 mcg | 10 mcg | | |
| | Over 71 years old | 15 mcg | 15 mcg | | |

| | | | |
|---|---|---|---|
| Folic acid | 400 mcg | 400 mcg food | 1,000 mcg synthetic |
| compound | 600 mcg for pregnant women | 200 mcg synthetic | |
| | | (400 mcg from | |
| | | supplements for | |
| | | women of child | |
| | | bearing age) | |
| Vitamin B₆ | 2 mg | 1.7 mg | 100 mg |
| Vitamin B₁₂ | 6 mcg | 2.4 mcg (over age 50, | Not Determined |
| | | supplements | |
| | | recommended) | |
| Boron | None | None | 20 mg (same for all |
| | | | adults) |

Information in this table was derived from the Council for Responsible Nutrition (2001)
*Reference Daily Intake (RDI) is the value established by the FDA for use in labeling
**Dietary Reference Intake (DRI) are the most recent set of dietary recommendations established by the Food and Nutrition Board of the Institute of Medicine
***Upper Limit (UL) is the upper limit of intake considered safe for use by adults (unless otherwise specified, the UL combines all potential sources of nutrient)

In some embodiments, the inventive formulations can preferably include amounts of one or more other vitamins or minerals that, in combination with folic acid compound, can promote healthy collagen production, promote general nutritional and dietary health, or can be specifically selected to treat or prevent another condition that may or may not relate to collagen.

The term “vitamin B₆” is used in the present description in a manner consistent with its understood meaning in the vitamin and medical arts. The term means compounds generally understood to provide vitamin B₆ efficacy in its administered form or as a derivative of the administered form. The compounds are understood to include pyridoxine compounds such as pyridoxine hydrochloride or any other of the vitamins of the B₆ complex (i.e., codecarboxylase, pyridoxal hydrochloride, or pyridoxamine dihydrochloride) or any precursors or analogues thereof which would give rise to vitamin B₆-like activity. Vitamin B₆ is a known cofactor in metabolizing homocysteine, and therefore can reduce serum homocysteine and further promote the production and maintenance of healthy collagen. A useful amount of vitamin B₆ in combination with the folic acid compound and any other added vitamins or minerals can promote healthy collagen by facilitating homocysteine metabolization.

The daily oral dosage of vitamin B₆ administered according to the invention can be any useful and therapeutic amount, and in certain preferred embodiments of the invention can exceed daily recommended intakes (“DRIs”) that are conventional for vitamin B₆, especially for certain individuals and perhaps for certain individuals having specific conditions. The standard DRI for vitamin B₆ is 1.7 mg per day. Exemplary dosages according to the inventive methods and formulations can include at least or greater than 5 mg, 10 mg vitamin B₆ per day, and depending on the collagen-related condition being treated, up to and including 20, 50, or 100 milligrams vitamin B₆ per day. The amount of vitamin B₆ can be administered in a single dose or in more than one dose taken at the same or different times throughout the day.

The term “vitamin B12” is used in the present description in a manner consistent with its understood meaning in the vitamin and medical arts. The term means compounds generally understood to provide vitamin B₁₂ efficacy in its administered form or as a derivative of the administered form. The compounds are understood to include cobalamin compounds such as hydroxycobalamin, methylcobalamin, and cyanocobalamin or any other substances or any precursors or analogues thereof which would give rise to vitamin B₁₂-like activity. Vitamin B₁₂ is a known cofactor in metabolizing homocysteine, and therefore can reduce serum homocysteine and further promote the production and presence of healthy collagen. A useful amount of vitamin B₁₂, in combination with the folic acid compound and any other added vitamins or minerals, can promote healthy collagen by facilitating homocysteine metabolization.

The daily oral dosage of vitamin B₁₂ administered according to the invention can be any useful and therapeutic amount, and in certain preferred embodiments of the invention may exceed daily recommended intakes (“DRIs”) that are conventional for vitamin B,2, especially for certain individuals and perhaps for certain individuals having specific conditions. The standard DRI for vitamin B₁₂ is 2.4 mcg per day. Exemplary daily dosages of vitamin B₁₂, according to the formulations and methods of the invention, can be from about 20 mcg, 100 mcg, or 150 micrograms vitamin B₁₂ up to about 1000 micrograms B₁₂. The amount of vitamin B₁₂ may be administered in a single dose or in more than one dose taken at the same or different times throughout the day.

The term “vitamin A” is used in a manner consistent with its understood meaning in the vitamin and medical arts. The term means compounds generally understood to provide vitamin A efficacy in its administered form or a derivative of its administered form. The compounds are understood to include β-ionone derivates possessing qualitatively the biological activity of retinol, retinol, β-carotene, and salts thereof such as the acetate or palmitate salts or any precursors or analogues thereof which would give rise to vitamin A-like activity after administration. The amounts of vitamin A to be administered per day can be any useful amount, such as an amount in the range from about 2000 to 4000 IU. The selected amount of vitamin A may be administered in a single dose or in more than one dose taken at once or at different times throughout the day.

The term “vitamin C” is used in a manner consistent with its understood meaning in the vitamin and medical arts. The term means compounds generally understood to provide vitamin C efficacy in its administered form or a derivative of its administered form. Vitamin C compounds include vitamin C in any of its forms (e.g., salts of ascorbic acid) or any precursor or analogue which would give rise to vitamin C-like activity after administration. A preferred form of vitamin C is ascorbic acid. The amounts of vitamin C to be administered per day can be any useful amount, such as an amount in the range from about 50 to 1000 mg, preferably from 100, 150, or 250 mg vitamin C to 500 mg vitamin C. The selected amount of vitamin C may be administered in a single dose or in more than one dose taken at once or at different times throughout the day.

The term “vitamin D” is used in a manner consistent with its understood meaning in the vitamin and medical arts. The term means compounds generally understood to provide vitamin D efficacy in its administered form or a derivative of its administered form. Exemplary vitamin D compounds include vitamin D₁, vitamin D₂, vitamin D₃, or vitamin D₄, or any precursor or analogue to any of these vitamins which would give rise to vitamin D-like activity after administration. The amounts of vitamin D to be administered per day can be any useful amount, such as an amount in the range from about 200 to 600 IU (5 to 15 mcg). The selected amount of vitamin D may be administered in a single dose or in more than one dose taken at once or at different times throughout the day.

The term “vitamin K” is used in a manner consistent with its understood meaning in the vitamin and medical arts. The term means compounds generally understood to provide vitamin K efficacy in its administered form or a derivative of its administered form. Exemplary vitamin K compounds include vitamin K₁, vitamin K₂, vitamin K₃, vitamin K₄, vitamin K₅, vitamin K₆ and vitamin K₇, or any precursor or analogue to any of these vitamins (such as the naphthaquinones), which would give rise to vitamin K-like activity after administration. The amounts of vitamin K to be administered per day can be any useful amount, such as an amount in the range from about 20 to 200 mcg. The selected amount of vitamin K may be administered in a single dose or in more than one dose taken at once or at different times throughout the day.

Any other vitamin or mineral discussed herein are also is used in a manner consistent with their understood meanings in the vitamin and medical arts. The terms identify compounds generally understood to provide vitamin or mineral efficacy in an administered form or as a derivative of an administered form. Examples of these terms include calcium, magnesium, thiamine, riboflavin, boron, iron, and vitamin E. The selected amount of any one or more of these vitamins and minerals may be administered in a single dose or in more than one dose taken at once or at different times throughout the day.

Calcium is a mineral supplement that is available in a variety of different forms such as calcium carbonate (e.g., from oyster shell) (this is presently the least expensive form of calcium but is theorized to cause constipation—still calcium carbonate can be preferred because it is very active and therefore helps to reduce dosage, e.g., tablet size for easy swallowing); calcium citrate (more available than calcium carbonate, doesn't cause constipation); calcium bi-glycinate (may be more available than calcium citrate).

| CALCIUM SALT | % CALCIUM | COMMON PRODUCTS |
|---|---|---|
| Calcium Gluconate | 9% | 500 mg tablets (45 mg elemental calcium) |
| Calcium Lactate | 13% | 650 mg tablets (84.5 mg elemental calcium) |
| Calcium Citrate | 21% | Citracal - 200 mg elemental calcium |
| Calcium Acetate | 25% | PhosLo 667 mg (169 mg elemental calcium) |
| Tricalcium Phosphate | 39% | Posture - 600 mg elemental calcium |
| Calcium Carbonate | 40% | Os-Cal - 500 mg elemental calcium (1,250 mg) |
| | | Caltrate - 600 mg elemental calcium (1,500 mg) |

Calcium supplementation can be included in the formulations and methods of the invention to provide a formulation that, in addition to promoting healthy collagen, can maintain healthy bones, in particular in postmenopausal women. Increased calcium intake has been directly correlated with increased bone mass. While results from individual studies are conflicting, it does not appear that there are significant differences between the various salt forms in terms of absorption or bioavailability—the total amount of elemental calcium is the most significant factor. Including calcium carbonate, for example at 750 mg (300 mg elemental calcium) per dosage formulation can reduce or minimize tablet size while still providing and 45% of the DRI for calcium when taken as two dosages or twice daily. Thus, exemplary daily dosages of calcium can be in the range from about 200 mg to about 1000 mg elemental calcium per day.

The inventive methods and formulation can also include administering magnesium. Magnesium can be available as any one or more of magnesium chloride, magnesium aspartate, magnesium oxide, magnesium succinate, magnesium gluconate, magnesium lactate, magnesium citrate, and as amino acid chelates. Magnesium oxide has the highest percent of elemental magnesium, and therefore can be preferred to minimize tablet size.

| MAGNESIUM SALT | % MAGNESIUM | COMMON PRODUCTS |
|---|---|---|
| Magnesium Gluconate | 5.9% | Magtrate 500 mg (29 mg elemental magnesium) |
| Magnesium Oxide | 60.3% | Uro-Mag 140 mg (84.5 mg elemental magnesium) |
| | | MagOx 400 mg (241 mg elemental magnesium) |
| Magnesium Chloride | 25.5% | Slo-Mag 535 mg (64 mg elemental magnesium) |
| | | (Slo-Mag uses chloride hexahydrate salt) |
| Magnesium Lactate | 12% | Mag-Tab SR (84 mg elemental magnesium) |
| Magnesium Citrate | 16.2% | Unknown |

The role of magnesium supplementation in the prevention of degenerative bone disease such as osteoporosis is less well established than for calcium. Although decreased bone mass is the hallmark of osteoporosis, qualitative changes in bone are also present. There is growing evidence that magnesium may play an important role in qualitative bone changes. In addition, adequate magnesium levels are necessary for proper calcium metabolism. Lastly, dietary intake studies consistently show that magnesium intake can sometimes or often be well below the established DRI, indicating that a number of individuals may be at risk for a magnesium deficiency. The upper limit for magnesium from supplements is established to minimize the incidence of diarrhea, which is the most common side effect of magnesium supplementation. Including 250 mg magnesium oxide (150 mg elemental magnesium) per tablet would minimize the tablet size while providing and 71% of the DRI (86% of the upper limit for supplements) for magnesium when two tablets are taken per day.

Exemplary daily dosages of riboflavin (vitamin B₂) can be in the range from about 3.5 to about 5 mg per day, but higher or lower amounts could also be useful.

Boron supplementation can be accomplished by administering boron, e.g., in the form of sodium borate, or potassium borate, boron citrate, boron aspartate, or boron glycinate (or other amino acid chelates). There is no recommended daily intake for boron, but some information suggests 2-3 mg daily for boron supplementation, and a normal daily intake through diet may be approximately, at maximum, 5 mg/day. Boron supplementation carries the following popular claims: prevents loss of magnesium, calcium, and phosphorus; prevents bone loss; and helps maintain healthy estradiol levels. Boron may have a role in vitamin D metabolism, which may indirectly impact bone development. A safe upper limit of 20 mg has been established for all adult populations (includes dietary and supplement amounts). The safe upper limit for pregnancy is 20 mg/day (based on animal reproduction studies). The maximum expected dietary consumption (including drinking water) is expected to be less than 5 mg/day. Exemplary daily dosages of boron according to the methods and compositions of the invention can be in the range from about 1 mg or 2 mg to about 5 mg per day, taken as a single dosage form or as two or more during a day.

Exemplary daily dosages of iron, according to the methods and formulations of the invention, can be in the range from about 10 to about 60 mg per day, but higher or lower dosages may also be useful.

As used herein, the term “formulation” refers to any sort of suitable dosage form used to deliver the compounds, as described herein, to a subject. Preferred formulations are oral dosage forms; however other formulations, for example, formulations for injection (e.g., subcutaneous, intramuscular, etc.), formulations for topical application, or formulations for rectal, nasal, or optical administration are contemplated and can be prepared and administered to a subject. As used herein “oral formulations” or “oral dosage forms” include any liquid composition, solid composition, or combination liquid/solid composition that can be suitable delivered to a subject. Formulations of the invention may be administered as oral dosage forms and may be. These oral dosage forms include solid dosage forms, for example, tablets, capsules, including soft elastic capsules or hard elastic capsules having a solid or liquid fill, lozenges, chewable tablets or chewable capsules, and liquid dosage forms, for example, solutions, suspensions, dispersions or syrups. Preferred dosage forms for the multivitamin composition may be capsules, soft-gel capsules, or tablets.

The formulation can include additional inactive ingredients, including, but not limited to surfactants, cosolvents, and excipients.

Surfactants, such as hydrophilic and hydrophobic surfactants, can be included in the formulations. Particular surfactants can be used based on the on the overall composition of the formulation and the intended delivery of the formulation. Useful surfactants include polyethoxylated fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, polysaccharide esters, polyethylene glycol alkyl phenols, polyoxyethylene-polyoxypropylene block copolymers, sorbitan fatty acid esters, lower alcohol fatty acid esters, ionic surfactants, and mixtures thereof.

The formulations can also include cosolvents such as alcohols and polyols, polyethylene glycols ethers, amides, esters, other suitable cosolvents, and mixtures thereof. The formulations can also include excipients or additives such as sweeteners, flavorants, colorants, antioxidants, preservatives, chelating agents, viscomodulators, tonicifiers, odorants, opacifiers, suspending agents, binders, and mixtures thereof.

Useful dosage forms can be prepared by methods and techniques that will be well understood by those of skill in the vitamin arts and may include the use of additional ingredients in producing tablets, capsules, or liquid dosage forms.

The formulations of the invention can be administered to promote collagen production in individuals who have or do not have a collagen-related condition, e.g., treat or prevent a collagen-related condition, or to otherwise facilitate healthy collagen production and healthy collagen tissue, for example, by maintaining healthy collagen tissue. Examples of methods according to the invention include the following:

Degenerative Tissue Conditions

A type of collagen-related condition is a class of degenerative bone or tissue diseases including degenerative bone disease, specifically including osteoporosis, as well as other degenerative diseases such as arthritis and Alzheimer's disease. According to the invention, the folic acid-containing formulations can be administered daily to promote collagen production and treat or prevent degenerative diseases such as osteoporosis, arthritis, and Alzheimer's disease, e.g., improve an existing degenerative condition, prevent further degeneration, or prevent degeneration before it occurs.

The dosage methods and formulations of the invention used to treat or prevent a degenerative bone or tissue disease or condition can preferably include a daily dosage of a folic acid compound in an amount that is greater than the DRI, e.g., greater than 400 mcg per day, preferably greater than 500 mcg per day, e.g., greater than 1000 or 2000 mcg per day. Such an amount can be administered, for example, in a single oral dosage or with two or more dosages taken at the same time or different times throughout the day. For example, a tablet of the invention may contain 1.1 mg of folic acid compound and two can be taken daily to provide 2.2 mg per day folic acid compound, to treat or prevent a degenerative bone or tissue disease.

In some embodiments, preferably, useful multivitamins for treating or preventing degenerative diseases such as osteoporosis, arthritis, and Alzheimer's disease, contain vitamin B₆, e.g., to provide a daily dosage of an amount in the range from about 5, 10, or 20 mg to about 50 mg per day vitamin B₆; and vitamin B₁₂, e.g., to provide a daily dosage of an amount in the range from about 100 or 150 mcg to about 1000 mcg per day vitamin B₁₂. Useful multivitamins for treating or preventing degenerative diseases such as osteoporosis, arthritis, and Alzheimer's disease, can also include one or more additional vitamins and minerals such as calcium, e.g., to provide a daily dosage the range from about 300 to about 1500 mg per day elemental calcium, e.g., from about 1000 mg to 1500 mg per day; magnesium, e.g., to provide a daily dosage in the range from about 25 to about 400 mg per day elemental magnesium; vitamin D (e.g., vitamin D₃), e.g., to provide a daily dosage in the range from about 5 to about 25 mcg vitamin D; boron, e.g., to provide a daily dosage in the range from about 1 to about 5 mg boron; as well as any other useful vitamins or minerals.

Wound Healing

Another example of a type of collagen-related condition includes tissue wound such as bone, skin, muscle, tendon, ligament, cartilage, or other tissue wounds. The compositions of the invention can be administered to facilitate wound healing generally by giving daily oral dosages as described herein to an individual having a skin wound, a muscle wound, a tendon or ligament wound, or another tissue wounds whose healing can be facilitated by healthy collagen production. The method includes administering formulations as described for general wound healing such as the treatment of cuts, bruises, lacerations, contusions, muscle or cartilage tears, skin sores, or other similar or related tissue wounds. In preferred embodiments the formulations are administered orally or topically. The method also includes administering the compositions for wound healing following surgical or other invasive or traumatic procedures, for example to facilitate healing of skin or muscle damaged as the result of a surgical incision required during a surgical procedure, or to facilitate healing of a specific muscle, bone, or other tissue that was the subject of the surgical procedure, e.g., a bone, muscle, or a ligament or other tissue that was repaired by the surgical procedure.

The dosage methods and formulations of the invention used to treat tissue wounds can preferably include an amount of a folic acid compound that is greater than the DRI, e.g., in an amount greater than 400 mcg per day, preferably greater than 500 mcg per day, e.g., greater than 1000 or 2000 mcg per day. Such an amount can be administered in a single oral dosage or two or more dosages taken at the same time or different times throughout the day. For example, a tablet of the invention may contain 1 mg of folic acid compound to be administered once a day.

In another embodiment, useful multivitamins for treating tissue wounds contain vitamin B₆, e.g., to provide a daily dosage in the range from about 5 to about 50 or 100 mg vitamin B₆ daily; and vitamin B₁₂, e.g., to provide a daily dosage in the range from about 20 to about 1000 mcg vitamin B₁₂ daily. Useful multivitamins for treating tissue wounds can also include one or more additional vitamins and minerals such as vitamin C, e.g., to provide a daily dosage of an amount in the range from about 100 to about 500 mg vitamin C daily; vitamin E, e.g., to provide a daily dosage of an amount in the range from about 10 to about 50 IU Daily; thiamine, e.g., to provide a daily dosage of an amount in the range from about 5 to about 20 mg thiamine daily; riboflavin, e.g., to provide a daily dosage of an amount in the range from about 5 to about 20 mg riboflavin daily; niacin, e.g., to provide a daily dosage of an amount in the range from about 10 to about 50 mg niacin daily; as well as any other useful vitamins or minerals including biotin, panthothenic acid, copper, zinc, etc.

Pre-natal Health and Fetal Development

Pre-natal health and fetal development can also relate to collagen, and can be promoted or improved by a diet that facilitates healthy collagen production. According to the invention, folic acid-containing compositions can be orally administered daily to promote collagen production in pregnant women to promote pre-natal health and development of fetal bone, muscle, and other tissue.

The dosage methods and formulations for pre-natal administration of folic acid-containing compositions according to the invention can preferably include an amount of a folic acid compound that is greater than the DRI, e.g., greater than 400 mcg per day, preferably greater than 500 or 600 mcg per day, e.g., greater than 1000 or 2000 mcg per day up to 5 mg or 10 mg per day of the folic acid compound. Such an amount can be administered in a single oral dosage or two or more dosages taken at the same time or different times throughout the day. For example, a tablet of the invention may contain greater than 1 mg of folic acid compound, preferably 2 mg, or more, to be taken once daily.

In another embodiment, useful multivitamins for pre-natal administration can contain vitamin B₆, e.g., to provide a daily dosage of an amount in the range from about 5 to about 100 mg vitamin B₆ per day (which can be useful also in reducing nausea/vomiting during pregnancy); and vitamin B₁₂, e.g., to provide a daily dosage of an amount in the range from about 15 to about 100 mcg vitamin B₁₂ per day, e.g., from about 40 to 75 mcg per day. Formulations according to the invention for pre-natal administration can also include one or more additional vitamins and minerals such as vitamin A, e.g., to provide a daily dosage of an amount in the range from about 2000 to about 5000 IU vitamin A per day; vitamin C, e.g., to provide a daily dosage of an amount in the range from about 125 to about 300 mg vitamin C; vitamin D, e.g., to provide a daily dosage of an amount in the range from about 200 to about 600 IU vitamin D per day; vitamin K, e.g., to provide a daily dosage of an amount in the range from about 20 mcg to about 200 mcg vitamin K per day; thiamine, e.g., to provide a daily dosage of an amount in the range from about 3.5 to about 5 or 10 mg thiamine per day; riboflavin, e.g., to provide a daily dosage of an amount in the range from about 3.5 to about 5 or 10 mg riboflavin per day; calcium, e.g., to provide a daily dosage in the range from about 200 to about 800 mg elemental calcium per day; magnesium, e.g., to provide a daily dosage of an amount in the range from about 25 to about 200 mg elemental magnesium per day; as well as any other useful vitamins or minerals.

Dermatological Health and Treatment or Prevention of Skin Conditions

Collagen is also important to dermatological health, e.g., healthy skin. According to the invention, healthy skin can be promoted by facilitating healthy collagen production, and skin conditions may be treated or prevented by facilitating healthy collagen production. According to the invention, folic acid-containing compositions can be administered daily to promote collagen production and treat or prevent skin conditions or to promote healthy skin. Preferred administration routes are oral and topical administration.

The dosage methods and formulations of the invention used to promote healthy skin or treat or prevent skin conditions can preferably include an amount of a folic acid compound that is greater than the DRI, e.g., greater than 400 mcg per day, preferably greater than 500 mcg per day, e.g., greater than 1000 or 2000 mcg per day, up to 5 or 10 mg per day. Such an amount can be administered in a single oral dosage or two or more pills taken at the same time or different times throughout the day. For example, a tablet of the invention may contain 1 mg of folic acid compound and can be taken once daily.

In another embodiment, useful multivitamins for promoting healthy skin or treating or preventing skin conditions can contain vitamin B₆, e.g., to provide a daily dosage of an amount in the range from about 5 to about 100 mg vitamin B₆ per day; and vitamin B₁₂, e.g., to provide a daily dosage of an amount in the range from about 20 to about 1000 mcg or 1 mg vitamin B₁₂ per day. Multivitamins according to the invention for promoting healthy skin or treating or preventing skin conditions can also include one or more additional vitamins and minerals such as vitamin A, e.g., to provide a daily dosage of an amount the range from about 2000 to about 4000 IU vitamin A per day; vitamin C, e.g., to provide a daily dosage of an amount in the range from about 125 to about 500 or 1000 mg vitamin C; as well as any other useful vitamins or minerals.

The described multivitamin formulations of this disclosure contain vitamins and minerals that do not require regulatory approval. The multivitamin formulations do not include aspirin or other pharmaceutical substances that in combination with the described vitamins and minerals would require regulatory approval for sale or use. The vitamins and minerals may be included in prescription or non-prescription amounts.

In one aspect, the formulations described herein can be prepared and used for treatment or prevention of diseases or conditions that relate to collagen, such as those described above. The formulations of the invention can include sufficient amounts of folic acid compound to facilitate healthy collagen production for treating or preventing the described conditions or for otherwise promoting healthy collagen for facilitating the production of healthy tissue that contains collagen. In some embodiments, the formulations preferably include therapeutic amounts of vitamin B₁₂ and vitamin B₆. In other embodiments, the formulations can include additional vitamins and minerals that can treat or prevent a specific disease or condition or that can be administered for general nutritional value. Aspirin or other substances that in combination with the described vitamins and minerals would require regulatory approval for sale or use, are not part of the multivitamin compositions of the invention.

### EXAMPLES

Example OS for, e.g., Osteoporosis

Example OS is a multivitamin tablet formulation that combines substances that can be administered to treat or prevent degenerative diseases or degenerative conditions such as degenerative bone disease, osteoporosis, Alzheimer's disease, and arthritis. The formulations include folic acid, vitamin B₆, and vitamin B₁₂, to promote healthy collagen production, and additionally include calcium and magnesium to diminish or prevent bone loss. The following dosage form can be taken or prescribed as two tablets taken once daily.

| | DAILY DOSE | | AMOUNT PER TABLET | |
|---|---|---|---|---|
| FOLIC ACID | 2.2 | mg | 1.1 | mg |
| B₆ | 25 | mg | 12.5 | mg |
| B₁₂ | 500 | mcg | 250 | mcg |
| CALCIUM | 600 | mg | 300 | mg |
| MAGNESIUM | 300 | mg | 150 | mg |
| VITAMIN D₃ | 15 | mcg | 7.5 | mcg |
| BORON | 3 | mg | 1.5 | mg |

Ingredients of certain commercially available formulations are shown in the table below. The formulations have less folic acid, vitamin B₆, vitamin B₁₂, calcium, magnesium, and boron, as compared to the Example OS formulation.

| | | | CENTRUM ® | | | |
|---|---|---|---|---|---|---|
| | EXAMPLE OS | | SILVER | | TRI-BORON PLUS | |
| FOLIC ACID | 1.1 | mg | 400 | mcg | — | |
| B₆ | 12.5 | mg | 3 | mg | — | |
| B₁₂ | 250 | mcg | 25 | mcg | — | |
| CALCIUM | 600 | mg | 200 | mg | 250 | mg |
| MAGNESIUM | 175 | mg | 100 | mg | 125 | mg |
| VITAMIN D₃ | 7.5 | mcg | 10 | mcg | 2.5 | mcg |
| BORON | 1.5 | mg | 150 | mcg | 75 | mcg |

| | AMOUNT PER | DAILY CONSUMPTION | |
|---|---|---|---|
| NUTRIENT | TABLET | (ELEMENTAL AMOUNT) | RATIONALE |
| Calcium | 750 mg (300 mg | 600 mg elemental calcium | Ca carbonate provides highest % |
| Carbonate | elemental calcium) | (60% RDI) | elemental calcium; minimize |
| | | | tablet size |
| Magnesium | 250 mg (150 mg | 300 mg elemental | Mg oxide provides highest % |
| Oxide | elemental magnesium) | magnesium (75% RDI) | elemental Mg; minimize tablet |
| | | | size; below UL to minimize |
| | | | diarrhea |
| Folate | 1.1 mg | 2.2 mg | Promote healthy collagen |
| B₆ | 12.5 mg | 25 mg | Promote healthy collagen |
| B₁₂ | 250 mcg | 500 mcg | Promote healthy collagen |
| Boron | 1.5 mg | 3 mg | Amount from supplement plus |
| | | | common dietary intake well |
| | | | below UL |
| Vitamin D | 7.5 mcg | 15 mcg | Total daily dose equals DRI; |
| | | | well below UL |

### Example PN

EXAMPLE PN is a formulation that can be used to meet the nutritional needs of pregnant women in a convenient once daily vitamin satisfying all of the supplementation requirements for a pregnant woman. Prescribed as one tablet daily.

| INGREDIENT | EXAMPLE PN | | DRI PREGNANCY* | |
|---|---|---|---|---|
| VITAMIN A (β-carotene) | 2700 | IU | 2700 | IU |
| VITAMIN C | 150 | mg | 90 | mg |
| VITAMIN D | 400 | IU | 400 | IU |
| VITAMIN E | 30 | IU | 22 IU-33 IU | |
| VITAMIN K | 25 | mcg | 120 | mcg |
| THIAMINE (B₁) | 4 | mg | 1.2 | mg |
| RIBOFLAVIN (B₂) | 4 | mg | 1.3 | mg |
| NIACIN | 20 | mg | 16 | mg |
| VITAMIN B₆ | 20 | mg | 1.7 | mg |
| FOLIC ACID | 2.0 | mg | 400 | mcg |
| VITAMIN B₁₂ | 50 | mcg | 2.4 | mcg |
| BIOTIN | 30 | mcg | 30 | mcg |
| PANTHOTHENIC ACID | 10 | mg | 5 | mg |
| CALCIUM | 300 | mg | 1300 | mg |
| IRON (carbonyl) | 45 | mg | 18 | mg |
| PHOSPHOROUS | 100 | mg | 1250 | mg |
| IODINE | 150 | mcg | 150 | mcg |
| MAGNESIUM | 100 | mg | 400 | mg |
| ZINC | 15 | mg | 11 | mg |
| SELENIUM | 25 | mcg | 55 | mcg |
| COPPER | 0.5 | mg | 0.9 | mg |
| MANGANESE | 2.0 | mg | 2.3 | mg |
| CHROMIUM | 35 | mcg | 35 | mcg |
| MOLYBDENUM | 45 | mcg | 45 | mcg |
| DOCUSTATE SODIUM | 50 | mg | | |

*DRI values for pregnancy have been included when available.

As compared to certain typical multivitamin formulations said to be useful for prenatal nutritional supplementation, Example PN includes relatively higher daily dosages of folic acid, vitamin B₆, and vitamin B₁₂, vitamin C, thiamine, riboflavin calcium, and iron.

EXEMPLARY MULTIVITAMIN FORMULATIONS

| | | | PRENATE | | | |
|---|---|---|---|---|---|---|
| INGREDIENT | EXAMPLE PN | | ADVANCED | | PRECARE | |
| VITAMIN A | 2700 | IU | 5000 | IU | None | |
| VITAMIN C | 150 | mg | 60 | mg | 120 | mg |
| VITAMIN D | 400 | IU | 400 | IU | 400 | IU |
| VITAMIN E | 30 | IU | 30 | IU | 30 | IU |
| VITAMIN K | 25 | mcg | 25 | mcg | None | |
| THIAMINE (B₁) | 4 | mg | 1.5 | mg | 3 | mg |
| RIBOFLAVIN (B₂) | 4 | mg | 1.7 | mg | 3.4 | mg |
| NIACIN | 20 | mg | 20 | mg | 20 | mg |
| VITAMIN B₆ | 20 | mg | 2 | mg | 3 | mg |
| FOLIC ACID | 2.0 | mg | 1000 | mcg | 1000 | mcg |
| VITAMIN B₁₂ | 50 | mcg | 6 | mcg | 30 | mcg |
| BIOTIN | 30 | mcg | 30 | mcg | 15 | mcg |
| PANTHOTHENIC ACID | 10 | mg | 10 | mg | 10 | mg |
| CALCIUM | 300 | mg | 162 | mg | 40 | mg |
| IRON | 50 | mg | 18 | mg | 8 | mg |
| PHOSPHOROUS | 100 | mg | 109 | mg | None | |
| IODINE | 150 | mcg | 150 | mcg | 150 | mcg |
| MAGNESIUM | 100 | mg | 100 | mg | 100 | mg |
| ZINC | 15 | mg | 15 | mg | 15 | mg |
| SELENIUM | 25 | mcg | 20 | mcg | 10 | mcg |
| COPPER | 0.5 | mg | 2 | mg | 2 | mg |
| MANGANESE | 2.0 | mg | 2 | mg | None | |
| CHROMIUM | 35 | mcg | 120 | mcg | 15 | mcg |
| MOLYBDENUM | 45 | mcg | 75 | mcg | 15 | mcg |

| | | | | | |
|---|---|---|---|---|---|
| DOCUSTATE SODIUM | 50 | mg | 50 | mg | None |

### Example PS

EXAMPLE PS combines the benefits of vitamin B₁₂, vitamin B₆, and folic acid with additional vitamins and minerals for use in Pre/Post Surgery indications for treating and healing skin, muscle, or other tissue wounds, where healthy collagen production can facilitate such treatment or healing. The following exemplary formulation can be administered as one tablet daily.

| INGREDIENT | AMOUNT PER TABLET | | DRI | |
|---|---|---|---|---|
| FOLIC ACID | 1.0 | mg | 400 | mcg |
| VITAMIN B₆ | 10 | mg | 1.7 | mg |
| VITAMIN B₁₂ | 250 | mcg | 2.4 | mcg |
| VITAMIN C | 500 | mg | 90 | mg |
| VITAMIN E | 30 | IU | 22 IU-33 IU | |
| THIAMINE (B₁) | 10 | mg | 1.2 | mg |
| RIBOFLAVIN (B₂) | 5.0 | mg | 1.3 | mg |
| NIACIN | 20 | mg | 16 | mg |
| BIOTIN | 30 | mcg | 30 | mcg |
| PANTHOTHENIC ACID | 10 | mg | 5 | mg |
| COPPER | 1.0 | mg | 0.9 | mg |
| ZINC | 20 | mg | 40 | mg |

| | EXAMPLE | | STRESS | | NEPHROVITE | |
|---|---|---|---|---|---|---|
| INGREDIENT | PS | | TABS | | RX | |
| FOLIC ACID | 1.0 | mg | 400 | mcg | 1 | mg |
| VITAMIN B₆ | 10 | mg | 5 | mg | 10 | mg |
| VITAMIN B₁₂ | 250 | mcg | 12 | mcg | 6 | mcg |
| VITAMIN C | 500 | mg | 500 | mg | 60 | mg |
| VITAMIN E | 30 | IU | 30 | IU | None | |
| THIAMINE (B₁) | 10 | mg | 10 | mg | 1.5 | mg |
| RIBOFLAVIN (B₂) | 5 | mg | 10 | mg | 1.7 | mg |
| NIACIN | 20 | mg | 100 | mg | 20 | mg |
| BIOTIN | 30 | mcg | 45 | mg | 300 | mcg |
| PANTHOTHENIC ACID | 10 | mg | 20 | mg | 10 | mg |

| | | | | |
|---|---|---|---|---|
| COPPER | 1.0 | mg | None | None |
| ZINC | 11 | mg | None | None |

As compared to certain typical multivitamin formulations, Example PS includes a combination of relatively higher daily dosages of folic acid, vitamin B₆, and vitamin B₁₂, vitamin C, copper and zinc.

### Example CP

EXAMPLE CP is a multivitamin formulation designed to combine the benefits of relatively high amounts of vitamin B₁₂, vitamin B₆, and folic acid with additional vitamins and minerals for use as a convenient high-potency multivitamin/multimineral. The following formulation can be taken as one tablet daily.

| INGREDIENT | AMOUNT PER TABLET | | DRI | |
|---|---|---|---|---|
| VITAMIN A | 3000 | IU | 3000 | IU |
| VITAMIN C | 500 | mg | 90 | mg |
| VITAMIN D | 400 | IU | 600 | IU |
| VITAMIN E | 30 | IU | 22 IU-33 IU | |
| VITAMIN K | 120 | mcg | 120 | mcg |
| THIAMINE (B₁) | 10 | mg | 1.2 | mg |
| RIBOFLAVIN (B₂) | 5.0 | mg | 1.3 | mg |
| NIACIN | 20 | mg | 16 | mg |
| VITAMIN B₆ | 10 | mg | 1.7 | mg |
| FOLIC ACID | 1.0 | mg | 400 | mcg |
| VITAMIN B₁₂ | 250 | mcg | 2.4 | mcg |
| BIOTIN | 30 | mcg | 30 | mcg |
| PANTHOTHENIC ACID | 10 | mg | 5 | mg |
| CALCIUM | 300 | mg | 1300 | mg |
| IRON | 18 | mg | 18 | mg |
| PHOSPHOROUS | 100 | mg | 1250 | mg |
| IODINE | 150 | mcg | 150 | mcg |
| MAGNESIUM | 100 | mg | 400 | mg |
| ZINC | 11 | mg | 11 | mg |
| SELENIUM | 25 | mcg | 55 | mcg |
| COPPER | 0.5 | mg | 0.9 | mg |
| MANGANESE | 2.0 | mg | 2.3 | mg |
| CHROMIUM | 35 | mcg | 35 | mcg |
| MOLYBDENUM | 45 | mcg | 45 | mcg |

| LUTEIN | 250 | | mcg | | None | |
|---|---|---|---|---|---|---|
| | | | | | established | |
| | EXAMPLE | | | | THERAGRAN | |
| INGREDIENT | CP | | CENTRUM | | M | |
| VITAMIN A | 3000 | IU | 5000 | IU | 5500 | IU |
| VITAMIN C | 500 | mg | 60 | mg | 120 | mg |
| VITAMIN D | 400 | IU | 400 | IU | 400 | IU |
| VITAMIN E | 30 | IU | 30 | IU | 30 | IU |
| VITAMIN K | 120 | mcg | 25 | mcg | None | |
| THIAMINE (B₁) | 10 | mg | 1.5 | mg | 3 | mg |
| RIBOFLAVIN (B₂) | 5.0 | mg | 1.7 | mg | 3.4 | mg |
| NIACIN | 20 | mg | 20 | mg | 20 | mg |
| VITAMIN B₆ | 10 | mg | 2 | mg | 3 | mg |
| FOLIC ACID | 1.0 | mg | 400 | mcg | 400 | mcg |
| VITAMIN B₁₂ | 250 | mcg | 6 | mcg | 9 | mcg |
| BIOTIN | 30 | mcg | 30 | mcg | 15 | mcg |
| PANTHOTHENIC ACID | 10 | mg | 10 | mg | 10 | mg |
| CALCIUM | 300 | mg | 162 | mg | 40 | mg |
| IRON | 18 | mg | 18 | mg | 18 | mg |
| PHOSPHOROUS | 100 | mg | 109 | mg | None | |
| IODINE | 150 | mcg | 150 | mcg | 150 | mcg |
| MAGNESIUM | 100 | mg | 100 | mg | 100 | mg |
| ZINC | 11 | mg | 15 | mg | 15 | mg |
| SELENIUM | 25 | mcg | 20 | mcg | 10 | mcg |
| COPPER | 0.5 | mg | 2 | mg | 2 | mg |
| MANGANESE | 2.0 | mg | 2 | mg | None | |
| CHROMIUM | 35 | mcg | 120 | mcg | 15 | mcg |
| MOLYBDENUM | 45 | mcg | 75 | mcg | 15 | mcg |

| | | | | | |
|---|---|---|---|---|---|
| LUTEIN | 250 | mcg | 250 | mcg | None |

| | | | | |
|---|---|---|---|---|
| BORON | None | 150 | mcg | None |
| NICKEL | None | 5 | mcg | None |
| SILICON | None | 2 | mg | None |
| TIN | None | 10 | mcg | None |
| VANADIUM | None | 10 | mcg | None |

As compared to certain typical multivitamin formulations, Example CP includes relatively higher daily dosages of folic acid, vitamin B₆, vitamin B₁₂, vitamin C, vitamin K, thiamine, riboflavin, and calcium.

### Example D

EXAMPLE D contains a combination of essential vitamins/minerals for promoting healthy skin, e.g., for facilitating and maintaining healthy skin, or for treating or preventing a specific skin condition such as acne, rashes, etc. The following formulation can be taken as one tablet daily.

| INGREDIENT | AMOUNT PER TABLET | DRI |
|---|---|---|
| FOLIC ACID | 1.0 mg | 400 mcg |
| VITAMIN-A (βcarotene) | 3000 IU | 3000 IU |
| VITAMIN B₆ | 10 mg | 1.7 mg |
| VITAMIN B₁₂ | 250 mcg | 2.4 mcg |
| VITAMIN C | 500 mg | 90 mg |
| VITAMIN E | 200 IU | 22 IU-33 IU |
| RIBOFLAVIN (B₂) | 5.0 mg | 1.3 mg |
| Niacin | 20 mg | 16 mg |
| Biotin | 30 mcg | 30 mcg |
| Selenium | 25 mcg | 55 mcg |
| Zinc (sulfate) | 30 mg | 15 mg |
| INGREDIENT | EXAMPLE D | DERMAVITE |
| FOLIC ACID | 1.0 mg | 400 mcg |
| VITAMIN-A (βcarotene) | 3000 IU | 3,500 IU |
| VITAMIN B₆ | 10 mg | 10 mg |
| VITAMIN B₁₂ | 250 mcg | None |
| VITAMIN C | 500 mg | 120 mg |
| VITAMIN E | 200 IU | 60 IU |
| RIBOFLAVIN (B₂) | 5.0 mg | 8.5 mg |
| NIACIN | 20 mg | None |
| BIOTIN | 250 mcg | 600 mcg |
| SELENIUM | 25 mcg | 50 mcg |
| ZINC (SULFATE) | 30 mg | 45 mg |
| COPPER | None | 2 mg |
| MANGANESE | None | 5 mg |
| CHROMIUM | None | 200 mcg |
| CALCIUM | None | 270 mg |
| SILICON | None | 20 mg |
| LYCOPENE | None | 5 mg |

### Example D Ingredient Rationale

| INGREDIENT | BENEFITS |
|---|---|
| FOLIC ACID | Healthy cell division and for protein and DNA synthesis. |
| VITAMIN A | Helps maintain the health of the skin and regulates |
| | skin cell growth. |
| VITAMIN B₆ | Important enzyme cofactor. |
| VITAMIN B₁₂ | Maturation of RBCs and DNA synthesis. |
| VITAMIN C | Essential for collagen production. Aids in tissue repair |
| | and cell regeneration. |
| VITAMIN E | Protects cells from oxidative damage and wrinkling. |
| VITAMIN B₂ | Deficiency may produce a defect in collagen synthesis. |
| NIACIN | Deficiency may produce dermatitis like symptoms. |
| BIOTIN | Necessary for metabolism and growth. Required by |
| | healthy nails and skin. |
| SELENIUM | Antioxidant that preserves tissue elasticity. |
| ZINC | Important in tissue and cell growth. |

1. A method of facilitating or maintaining collagen formation to treat or prevent a collagen-related condition, the method comprising orally administering a formulation comprising greater than 400 micrograms folic acid compound.

2. The method of claim 1 comprising orally administering a formulation comprising greater than 500 micrograms folic acid compound.

3. The method of claim 1 comprising orally administering formulation comprising greater than 1000 micrograms folic acid compound.

4. The method of claim 1 comprising administering the formulation to treat or prevent a condition selected from the group consisting of a degenerative bone disease, osteoporosis, arthritis, and Alzheimer's disease.

5. The method of claim 1 comprising administering the formulation to facilitate collagen formation, to treat or facilitate healing of a skin or muscle wound.

6. The method of claim 1 comprising administering the formulation to facilitate or maintain collagen formation to promote post-surgical skin or muscle tissue healing.

7. The method of claim 1 comprising administering the formulation to facilitate collagen formation and promote pre-natal health or fetal development.

8. The method of claim 1 comprising administering the formulation to facilitate collagen formation in the treatment of osteoporosis.

9. The method of claim 1 comprising administering the formulation to facilitate collagen formation in the treatment of a skin condition.

10. The method of claim 1 wherein the formulation comprises:
a) from about 0.5 to about 10 milligrams folic acid compound,
b) from about 10 to about 100 mg vitamin B₆,
c) from about 50 to about 1000 micrograms vitamin B₁₂, and
d) one or a combination of additional vitamins and minerals selected from the group consisting of:
i) from about 200 to about 1000 mg calcium;
ii) from about 100 to about 1000 milligrams vitamin C;
iii) from about 50 to about 200 micrograms vitamin K;
iv) from about 25 to about 250 milligrams magnesium;
v) from about 5 to about 10 micrograms vitamin D;
vi) from about 5 to about 15 milligrams thiamine;
vii) from about 4 to about 7 milligrams riboflavin;
viii) from about 1 to about 5 milligrams boron;
and combinations thereof.

11. The method of claim 1 wherein the formulation comprises:
a) from about 0.5 to about 5 milligrams folic acid compound;
b) from about 5 to about 50 milligrams vitamin B₆;
c) from about 50 to about 1000 micrograms vitamin B₁₂;
d) from about 200 to about 1000 mg calcium; and
e) from about 200 to about 1000 milligrams vitamin C.

12. The method of claim 11 wherein the formulation further comprises:
a) from about 50 to 200 micrograms vitamin K;
b) from about 3.5 to 15 milligrams thiamine; and
c) from about 3.5 to 15 milligrams riboflavin.

13. A method of facilitating or maintaining collagen formation, the method comprising orally administering a formulation comprising greater than 400 micrograms folic acid compound at least one time per day, to provide a daily dosage of greater than 800 micrograms folic acid compound per day.

14. The method of claim 13 wherein the formulation comprises:
a) from about 1 to about 10 milligrams folic acid compound;
b) from about 10 to about 50 mg vitamin B₆;
c) from about 50 to about 1000 micrograms vitamin B₁₂; and
d) one or more additional vitamins or minerals selected from the group consisting of:
i) from about 300 to about 1000 mg calcium;
ii) from about 25 to about 250 milligrams magnesium;
iii) from about 5 to about 15 micrograms vitamin D;
iv) from about 1 to about 5 milligrams boron;
and combinations thereof.

15. A method of treating or preventing degenerative bone disease, Alzheimer's disease, or arthritis, the method comprising administering a formulation comprising greater than 1 milligram folic acid compound.

16. The method of claim 15 comprising administering the formulation comprising from about 1 to about 10 milligrams folic acid compound two or more times per day to provide a daily dosage of greater than 2 milligrams folic acid compound per day.

17. The method of claim 15 wherein the formulation comprises vitamin B₆, vitamin B₁₂, calcium, magnesium, vitamin D, and boron.

18. The method of claim 15 wherein the formulation comprises magnesium oxide.

19. The method of claim 15 wherein the step of administering provides a daily dosage comprising:
a) from about 1 to about 10 milligrams folic acid compound;
b) from about 10 to about 50 mg vitamin B₆;
c) from about 100 to about 1000 micrograms vitamin B₁₂; and
d) one or more additional vitamins or minerals selected from the group consisting of:
i) from about 300 to about 2000 mg calcium;
ii) from about 25 to about 500 milligrams magnesium;
iii) from about 1 to about 5 milligrams boron; and
and combinations thereof.

20. A method of improving pre-natal health or fetal development, the method comprising orally administering a formulation comprising from about 1.5 to about 10 milligrams folic acid compound.

21. The method of claim 20 wherein the formulation comprises one or more additional vitamins selected from the group consisting of:
a) from about 125 to about 300 mg vitamin C;
b) from about 5 to about 100 mg vitamin B₆;
c) from about 15 to about 100 micrograms vitamin B₁₂;
and combinations thereof.

22. The method of claim 20 wherein the formulation comprises
a) from about 125 to about 300 mg vitamin C;
b) from about 5 to about 50 mg vitamin B₆;
c) from about 15 to about 100 micrograms vitamin B₁₂;
d) from about 3.5 to 5 mg thiamine;
e) from about 3.5 to 5 mg riboflavin;
f) from about 200 to about 1000 mg calcium;
g) from about 25 to about 75 mg iron; and
h) from about 25 to about 250 milligrams magnesium.

23. A method of promoting healthy skin, the method comprising administering a formulation comprising greater than 400 micrograms folic acid compound.

24. A method of promoting post-surgical health or recovery, the method comprising administering a formulation comprising from about 1 to about 10 milligrams folic acid compound and from about 20 to 500 micrograms vitamin B₁₂.

25. A method of promoting healing of a skin or muscle wound, the method comprising administering a formulation comprising from about 1 to 10 milligrams folic acid compound and from about 20 to 500 micrograms vitamin B₁₂.

26. A formulation consisting of:
a) at least about 0.5 milligrams folic acid compound;
b) at least about 10 mg of vitamin B₆;
c) at least about 50 micrograms of vitamin B₁₂;
d) at least one or more vitamins or minerals selected from the group consisting of:
i) at least about 200 to about 800 mg calcium;
ii) at least about 100 to about 1000 milligrams vitamin C;
iii) at least about 50 to about 200 micrograms vitamin K;
iv) at least about 25 to about 200 milligrams magnesium;
v) at least about 5 to about 10 micrograms vitamin D;
vi) at least about 5 to about 15 milligrams thiamine;
vii) at least about 4 to about 7 milligrams riboflavin; and
viii) at least about 1 to about 5 milligrams boron; and
e) inactive ingredients for the delivery of the formulation to a subject; wherein the formulation facilitates or maintains collagen formation when administered to a subject.

27. The method of claim 1 wherein the formulation comprises magnesium oxide.

28. The method of claim 8 wherein the formulation comprises magnesium oxide.

29. The method of claim 20 wherein the formulation comprises magnesium oxide.

30. An oral dosage form comprising:
a) from about 0.5 to about 10 milligrams folic acid compound,
b) from about 10 to about 100 mg vitamin B₆,
c) from about 50 to about 1000 micrograms vitamin B₁₂, and
d) magnesium oxide.

31. A method of treating osteoporosis comprising administering the dosage form of claim 30.
